(19) United States
(12) Patent Application Publication
(10) Pub. No.: US 2006/0195114 A1
(43) Pub. Date: Aug. 31, 2006

(54) Method of using a vertebral retainer-distracter

(76) Inventor: Rudolf Bertagnoli, Vienna (AT)

Correspondence Address: STITES & HARBISON PLLC, 1199 NORTH FAIRFAX STREET, SUITE 900, ALEXANDRIA, VA, 22314, US

(21) Appl. No.: 11/413,011

(22) Filed: Apr. 28, 2006

Related U.S. Application Data
(62) Division of application No. 10/622,803, filed on Jul. 21, 2003.

(51) Int. Cl. A61B 17/58

(52) U.S. Cl. 606/90

(57) ABSTRACT

A method for distracting at least two adjacent vertebrae and/or retaining them in a spaced apart position. Anchor screws are secured to the adjacent vertebrae and tubes of a retainer-distracter instrument frame are slid down over the anchor screws, after which the anchor screws are positively secured to the tubes of the frame. The frame includes a structure for moving the arms and hence the anchor screws and vertebrae toward and away from each other. After the anchor screws and frame are attached, a separate distracter distracts the vertebrae away from each other, whereupon the instrument acts only as a retainer to hold the vertebrae apart.

### CROSS REFERENCE TO RELATED APPLICATIONS

This application is a divisional of application Ser. No. 10/622,803, filed Jul. 21, 2003 (which is hereby incorporated by reference).

### FIELD OF THE INVENTION

This invention relates to the field of intervertebral implants, and it relates in particular to a method of using a retainer-distracter.

### BACKGROUND OF THE INVENTION

Spinal surgery often requires removal of the existing intervertebral disc tissue located between adjacent vertebrae and replacement thereof with an intervertebral implant which may take the form of a cage or other fusion device or an artificial disc which may be of the type which allows limited universal movement of the adjacent vertebrae with respect to each other.

In any event, it is necessary to initially separate the adjacent vertebrae from each other and to retain them apart prior to cleaning out the existing disc tissue and inserting the intervertebral implant.

It has been known heretofore to distract adjacent vertebrae away from each other using an instrument having a pair of anchor screws, one anchored in each of the adjacent vertebrae, wherein the anchor screws are received loosely in tubes of a frame member, which frame member and tubes are then manipulated to move the anchor screws, and hence the adjacent vertebrae, away from each other. However, this known instrument has the disadvantage that because of the loose engagement between the tubes of the frame member and the anchor screws, when exerting the necessary forces to separate the anchor screws and hence the adjacent vertebrae from each other, the anchor screws and tubes would become misaligned, causing the instrument to jam, after which it could not successfully perform its intended function of separating the adjacent vertebrae by a predetermined amount and then positively and accurately retaining them in the selected appropriate spaced apart position. In addition, utilizing this type of instrument as a distracter is possible only when the bone tissue is relatively hard. If it is not relatively hard, the forces exerted on the vertebrae by the anchor screws to cause distraction will cut through the vertebrae to which they are attached.

### SUMMARY OF THE INVENTION

It is a purpose of the present invention to provide improvements in the field of intervertebral implants, and in particular, instruments and methods for distracting adjacent vertebrae and retaining them in the distracted condition, and methods for using such instruments, which overcome the disadvantages of the prior art.

In accordance with a first aspect of the present invention, the retainer-distracter instrument includes a pair of anchor screws which are screwed into the adjacent vertebrae and a frame member having arms, each arm having a tube which encircles one of the anchor screws, wherein the arms are operatively connected together to permit movement of the arms towards and away from each other. In accordance with the present invention, a retaining structure is provided for tightly securing each of the anchor screws in its respective tube.

In accordance with one embodiment of the present invention, the tubes are opened to permit a portion of the anchor screws remote from the vertebrae engaging portion thereof to be accessible and retaining structures are secured thereon which, when secured, tighten the anchor screws relative to their respective tubes. With the two anchor screws thus tightly secured to their respective tubes of the frame member, movement of the arms of the frame member towards and away from each other effects a positive controlled accurate aligned movement of the anchor screws, and hence also of the adjacent vertebrae, towards or away from each other. When using this instrument as a distracter, the arms would be moved apart, whereupon the arms would be kept parallel to each other and in the present invention, would also keep the tubes and their respective anchor screws in parallel planes. Of course using the present instrument as a distracter is possible only if the bone tissue of the adjacent vertebrae is relatively hard. If the bone tissue is not hard, the anchor screws, when forced apart to effect distraction, would tend to cut through the vertebrae tissue.

The frame member may be mounted on a bar for movement of the arms along the bar towards and away from each other, although the arms may be connected together with other mechanisms which permit their movement towards and away from each other. The ends of the anchor screws remote from the vertebrae may project out from the ends of the tubes or they may be located within the tubes, wherein the ends of the tubes would be sufficiently wide at the tops thereof to receive retaining structures. The retaining structures could include a retaining nut threaded onto the end of its respective anchor screw, or it could comprise any other retaining structure such as a resilient cap, a bayonet joint, or the like. While the instrument of the present invention is described with respect to two arms, and hence two anchor screws, it is also possible for the instrument of the present invention to have three arms securing three anchor screws in order to separate two vertebrae away from a third vertebrae located between the two said vertebrae in order to open up two adjacent intervertebral spaces.

In accordance with another aspect of the present invention, there is provided an improved method for separating adjacent vertebrae and retaining them in a spaced apart condition. In accordance with this method, one first attaches the retainer-distracter instrument (also referred to below as a retainer instrument) in the manner described above. However, in accordance with the present method, this retainer instrument is not used to perform distraction. Rather, a conventional distracter, e.g., of the pliers type, is inserted into the intervertebral space and caused to move the adjacent vertebrae away from each other. The retainer instrument is constructed such that the arms, tubes and anchor screws can move freely away from each other. However, the arms, tubes and anchor screws are retained against movement towards each other. Thus, after the distracter has moved the adjacent vertebrae to a desired spaced apart distance, and the arms of the retainer instrument have moved to that position, the distracter instrument is removed, after which the retainer instrument now positively retains the adjacent vertebrae in that spaced apart condition in preparation for further steps which will culminate in insertion of the intervertebral implant.

The intervertebral implant is normally inserted from the patient's anterior moving towards the patient's posterior. However, it is to be understood that the implant, the instruments and the method can also be designed and arranged to insert the implant laterally, i.e., from the side. Although the terms “anterior” and “posterior” will sometimes be used in the conventional sense with respect to the patient's anatomy, for purposes of convenience, the invention will be described herein primarily with respect to more simple terminology which relates to the instruments and methods themselves. For example, in describing the invention, the terms “front” or “forward” mean the part of the instrument which faces toward the vertebrae or is moving in the direction of movement toward the vertebrae, while the words “back”, “rear” or “rearward” refer to the end of the instrument farthest from the vertebrae or moving away from the vertebrae.

Thus, it is an object of the present invention to provide a new and improved retainer-distracter instrument for preparation of an intervertebral space for receiving an intervertebral implant.

It is another object of the present invention to provide a new and improved method for separating adjacent vertebrae and retaining them in their spaced apart condition.

These and other objects of the present invention will be apparent from the detailed description to follow, together with the accompanying drawings.

### BRIEF DESCRIPTION OF THE DRAWINGS

Embodiments of the present invention will now be described by way of example with reference to the accompanying drawings, wherein:

FIG. 1 is a perspective view of an assembled instrument having the features of the present invention;

FIG. 2 is a perspective view of a frame;

FIG. 3 is a perspective view of an anchor screw;

FIG. 4 is a perspective view of a retaining nut;

FIG. 5 is a perspective view of an anchor screw driver;

FIG. 6 is an elevational view of the frame of FIG. 2, taken in the direction of the arrow A thereof;

FIG. 7 is a top plan view of a portion of FIG. 6;

FIG. 8 is a cross-sectional view taken along line 8-8 of FIG. 6;

FIG. 9 is a perspective view of a vertebral distracter;

FIG. 10 is an enlarged schematic view of the top of a anchor screw and tube, showing modifications of the present invention;

FIG. 11 is a schematic view of a frame member having more than two arms;

FIG. 12 is a schematic view illustrating modifications of the present invention; and

FIGS. 13-16 show the steps in the method of using the instruments of FIGS. 1-12 to distract adjacent vertebrae and retain them in the distracted position.

### DETAILED DESCRIPTION OF THE PREFERRED EMBODIMENTS

Referring to the figures, like elements are represented by like numerals throughout the several views.

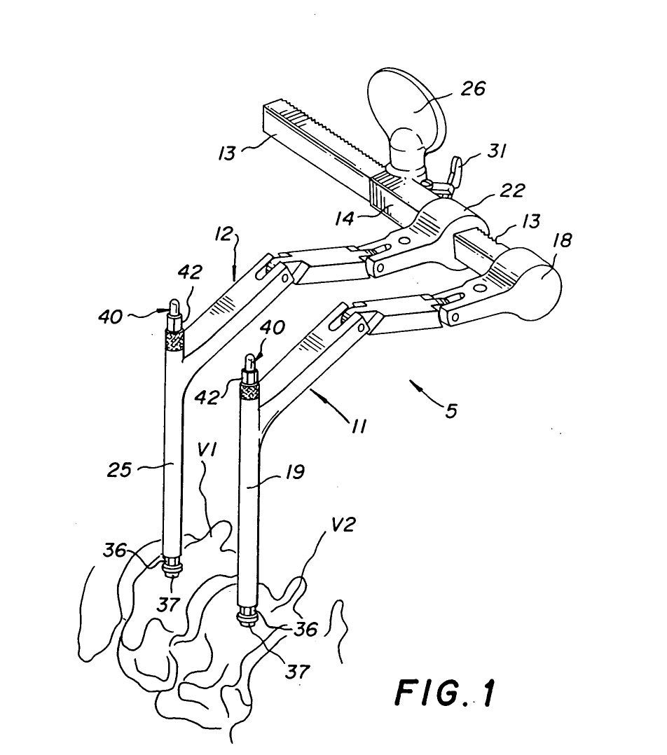

FIG. 1 illustrates a retainer-distracter instrument 5 (also referred to as a retainer instrument) having the features of the present invention. Instrument 5 can be used as a distracter, as such instruments have been used in the past, but now with the improvements of the present invention, as described below. In the alternative, the instrument 5 can be used solely as a retainer, in which case other structures would be provided for effecting distraction of the adjacent vertebrae to which the instrument 5 is attached. FIGS. 1-12 show the tools used with and the component parts of the instrument 5 of FIG. 1 as well as modifications.

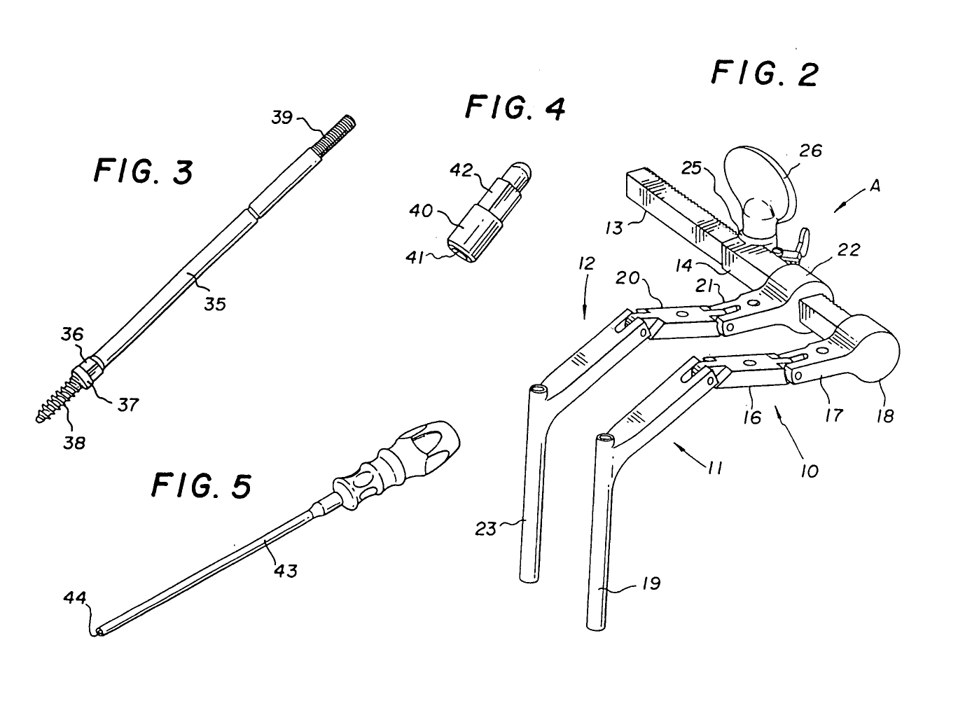

FIG. 2 shows the frame 10 which has first and second arms 11 and 12. A toothed rod 13 is telescopically received within a sleeve 14 and has a rack formed by teeth 15. The arm 11 has first and second articulated parts 16 and 17, the latter of which is integral with a hub 18 which is fixed onto the end of toothed rod 13. The arm 11 includes a hollow tube 19. The arm 12 includes articulated parts 20 and 21, the latter of which is integral with a hub 22 which is fixed to the end of sleeve 14. The arm 12 includes a hollow tube 23.

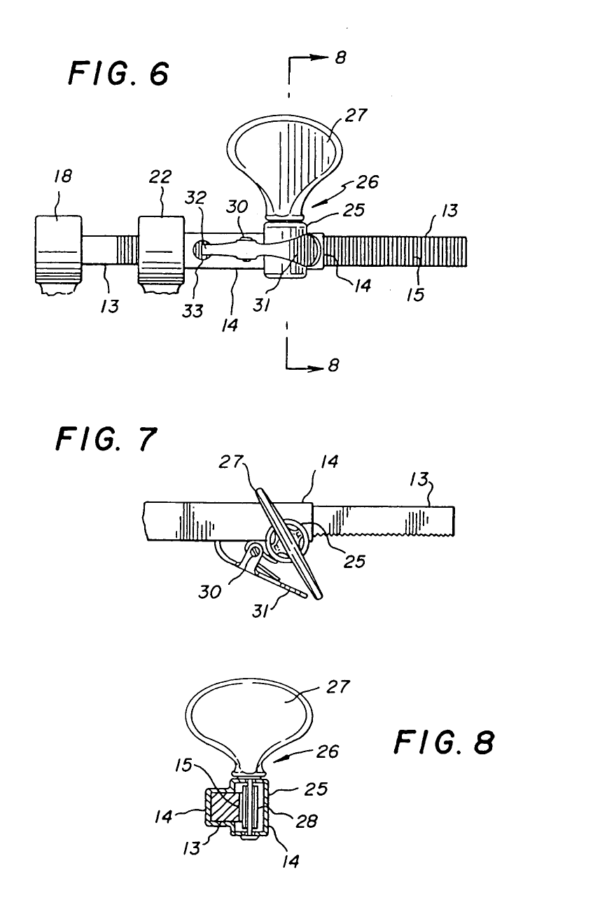

Referring primarily to FIGS. 6-8, a cylinder 25 is fixed to the periphery of sleeve 14. A thumb screw 26 has a thumb handle 27 and teeth 28 which are located in the cylinder 25 and engage the teeth 15 of the rod 13.

Fixed to the side of cylinder 25 is a spring 30 which engages a lever 31 so as to urge the lever 31 clockwise, as viewed from above in FIG. 7, about the axis of the spring 30, thereby causing the tip 32 of the level 31 to engage the teeth 15 of rod 13 through an aperture 33 in the sleeve 14.

FIG. 2 illustrates the frame 10 without the anchor screws or retaining nuts. FIG. 3 illustrates an individual anchor screw which comprises a polygonal nut portion 36, forward of which is a flange 37 which limits movement of the anchor screw into the bone, and forward of the flange 37 is the threaded front end of the anchor screw which, in use, is anchored into the vertebrae. At its opposite, rear end, the anchor screw 35 is threaded as shown at 39. FIG. 4 illustrates a retaining nut 40 which is interiorly threaded at 41 to match the threads 39 and has a polygonal nut portion 42 which is similar in dimensions to the nut portion 36 of the anchor screw 35. FIG. 5 illustrates an anchor screw driver 43, which is hollow and socket 44 at the forward end which is polygonal-shaped to match the shape of nut portions 36 and 42.

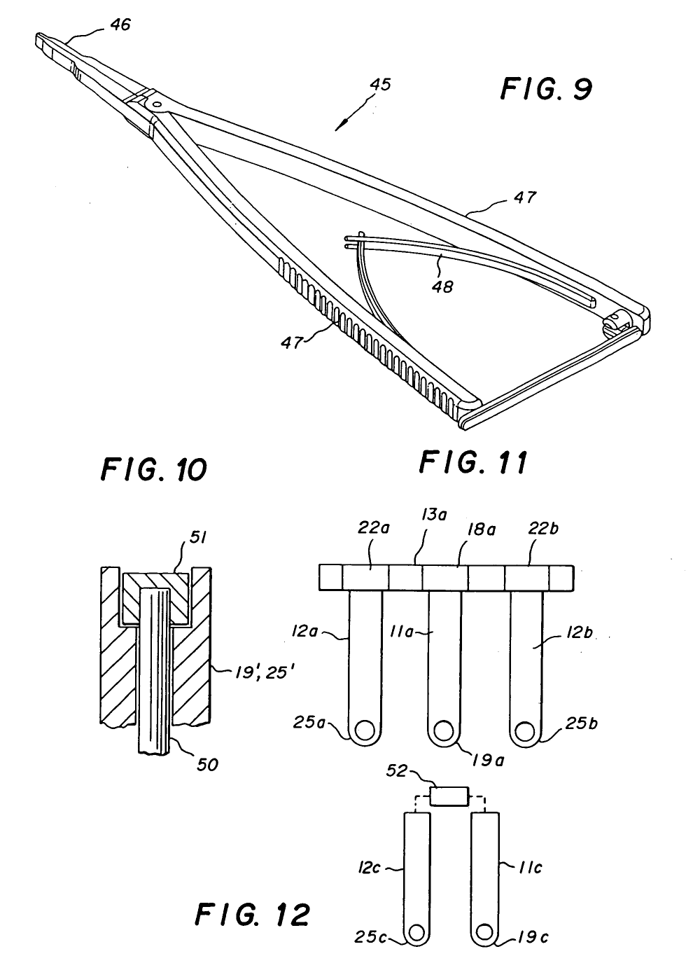

FIG. 9 illustrates a conventional distracter or spreader instrument 45 having a forward end 46 which can be inserted between adjacent vertebrae which have not yet been fully separated from each other. Handles 47 of this distracter are urged apart by a spring 48. To distract adjacent vertebrae from each other, the end 46 would be placed between adjacent vertebrae and the two portions of handle 47 would be moved together opposing the separating force of spring 48.

FIGS. 10-12 illustrate modifications of the present invention. In FIG. 1, the retaining structure is provided in the form of retaining nuts 46 which screw down onto threaded ends of the anchor screws 35 which project up through the open ends of the tubes 19 and 25. Instead of projecting up through the open ends of tubes 19 and 25, the tubes themselves, as shown at 19′,25′ in FIG. 10 may have enlarged openings for receiving retaining structures below the upper ends thereof. In this case the anchor screw, indicated at 50, would terminate within the enlarged area, below the top of the tubes 19′,25′. FIG. 10 also illustrates schematically another variation of the present invention wherein a retaining structure 51, shown schematically, represents other suitable securing structures, for example a resilient cap or a cap which engages the top of the anchor screw with a bayonet-type joint. Referring to FIG. 11, the instrument of the present invention may have three arms 11a, 12a and 12b with tubes 19a, 25a and 25b formed thereon, each for receiving an anchor screw (not shown) for the purpose of spreading apart two vertebrae on opposite sides of a middle vertebrae, thereby separating two adjacent intervertebral spaces. In this case the instrument including the central arm 11a and its hub 18a and the arm 12a and hub 22a to the left thereof on the crossbar 13a would be essentially identical to the frame structure as shown in FIG. 1. In addition, attached to the left of the fixed hub 18a would be the additional arm 12b and hub 22b. Crossbar 13a would have a structure to the right of hub 18a which would be the mirror image of the structure to the left as shown in FIGS. 1-8. Thus, the anchor screw of the arm 11a would grasp the central vertebrae of the three involved vertebrae and the arms 12a and 12b and their associated anchor screws would move their respective vertebrae away from the vertebrae anchored by the arm 11a.

FIG. 12 illustrates another modification of the present invention wherein the arms 11c and 12c which form the framework and include anchor screw engaging tubes 19c and 25c at the ends thereof may be connected together by any suitable means 52, other than the specific structure shown in FIGS. 1-8, which are capable of securing the arms and moving them towards and away from each other.

In using the instrument of the present invention, and referring to FIG. 1, a pair of anchor screws 35 would first be screwed into adjacent vertebrae V1 an V2. The hollow screw driver 43 would be moved over the entire length of each anchor screw 35 until its polygonal-shaped socket 44 engages the nut portion 36, whereupon the screw driver 43 would be turned to drive the threads 38 into the vertebrae V1 and V2 until the flange 37 engages the bone. After both anchor screws have been screwed into the vertebrae V1 and V2, the frame 10 is brought over onto the anchor screws with the tubes 19 and 23 encircling the anchor screws 35. The dimensions of the tubes 19 and 23 are such that their lower ends will engage and cannot move beyond the nut portions 36. In this position, the threaded upper ends 39 both project upwardly through openings in the top of tubes 19 and 23. The retainer nuts 40 are then threaded onto the exposed threaded ends 39 of the anchor screws 35 after which the screw driver 43 is used to engage its polygonal socket 44 onto the nut portions 42 to securely tighten the retainer nuts 40 and thereby securely tighten the anchor screws 35 within their respective tubes 19 and 23. At this point the anchor screws and the frame 10 form a very secure, tight, unitary unit.

With the anchor screws thus secured on the frame 10, the instrument is ready for movement of the arms 11 and 12 away from each other to distract the vertebrae V1 and V2.

If the instrument 5 is used strictly as a distracter, one would then grasp the handle 27 of thumb screw 26 and turn it counterclockwise as viewed from above in FIG. 7. The teeth 28 on the thumb screw 26 would then engage the teeth 15 on rod 13 to move the sleeve 14 to the right along the rod 13, thus separating the hub 22 from the hub 18 and thus moving the arm 12 away from the arm 11, thus separating the vertebrae V1 from the vertebrae V2, opening up the intervertebral space. During this movement of the sleeve 14 to the right along rod 13, the tip 32 of the lever 31, which is spring biased against the teeth 15 by spring 30, will ride over the teeth 15, thus allowing such movement of the sleeve 14 to the right along the rod 13. Of course during this same time the tip 32 will engage the teeth 15 to prevent movement of the sleeve 14 in the opposite direction, i.e., to the left. Thus, during this distracter movement, the sleeve 14 cannot move to the left unless one intentionally presses the right hand end of lever 31, turning it counterclockwise about its spring 30 to lift the tip 32 out from between the teeth 15 of rod 13.

In accordance with a method of the present invention, for distracting the adjacent vertebrae and retaining them apart, with the two vertebrae V1 and V2 in their closest position, one would perform all distraction with an instrument separate from the instrument 5, for example a distracter or spreader 45 as shown in FIG. 9. Using this distracter, and assisted by the rigid connection between the anchor screws 35 and the frame 10, as the distracter separated the vertebrae V1 and V2 from each other, the arm 12, including its hub 22 and its sleeve 14 would move freely away from the arm 11 as the tip 32 of lever 31 would simply ride over the crest of teeth 15. Once the desired separation had been achieved, the instrument 5 would act as a retainer to prevent the two arms 11 and 12 from moving towards each other as the tip 32 of lever 31 engaged the teeth 15 of rod 13.

If the instrument were used to separate two adjacent intervertebral spaces, the instrument shown in FIG. 11 would be attached to three adjacent vertebrae in essentially the same manner as described above with respect to the method of separating only two adjacent vertebrae. In this case, however, after the anchor screws were attached to the three adjacent vertebrae, it would be preferable to separate the vertebrae to form the intervertebral spaces first on one side, and then on the other side.

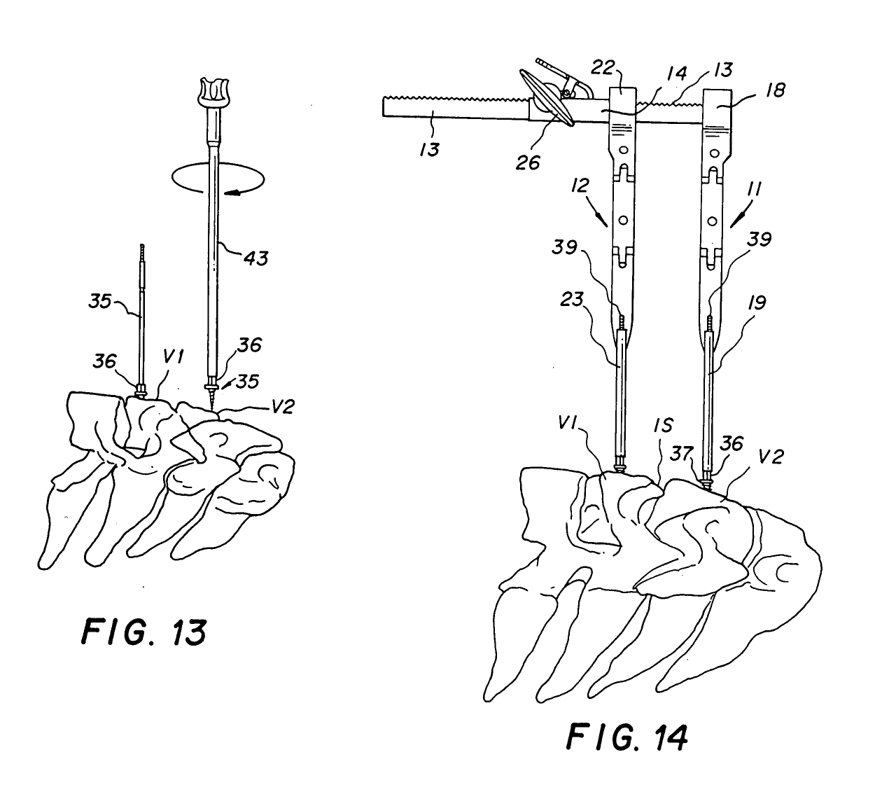

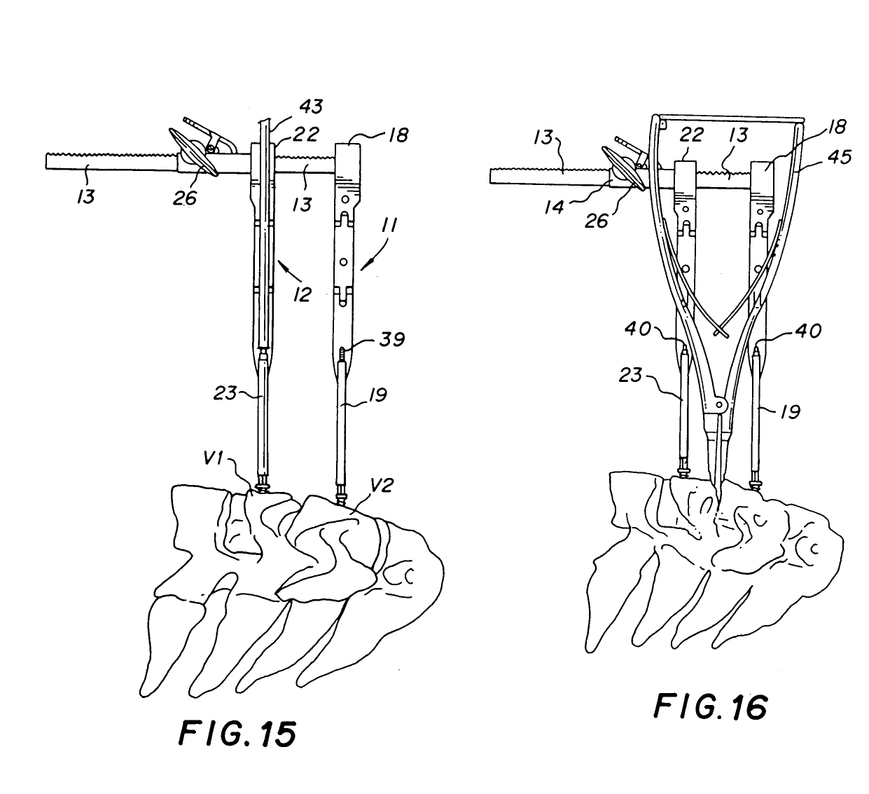

The method of operation of the present invention is further described with reference to FIGS. 13-16. FIG. 13 illustrates inserting the anchor screws 35 into adjacent vertebrae V1 and V2, using the anchor screw driver 43. FIG. 14 illustrates the instrument after the tubes 19 and 23 of the frame 10 have been slid over the anchor screws 35 to the point where the upper ends 39 of the anchor screws project through the tops of tubes 19 and 23. Next, referring to FIG. 5, the retainer nuts 40 are threaded onto the ends 39 of the upper exposed ends of the anchor screws 35 and for good tightness, are secured thereon by the anchor screw driver 43, wherein the polygonal socket 44 thereof engages the nut portions 42 of the retainer nuts. Finally, FIG. 16 illustrates the distraction of the adjacent vertebrae V1 and V2 using the distracter 45 of FIG. 9. As noted above, during this distraction, the arms 11 and 12 will move apart as the sleeve 14 rides along the rod 13, and when the desired limit position is reached, the tip 32 of lever 31 will engage the teeth 15 on rod 13 to prevent movement of the arm 12 toward the arm 11. In this position, the instrument 5 positively and securely retains the adjacent vertebrae V1 and V2 in their appropriate distracted position for further steps of the implant insertion procedure.

Although the invention has been described in considerable detail with respect to preferred embodiments, it will be apparent that the invention is capable of numerous modifications and variations, apparent to those skilled in the art, without departing from the spirit and scope of the claims.

What is claimed is:

1. A method for separating adjacent vertebrae from each other and maintaining them in a spaced apart condition, comprising the steps of:
attaching anchor screws to at least two adjacent vertebrae, which anchor screws are operatively mounted to a frame to be freely moveable away from each other but not freely moveable towards each other,
separating the adjacent vertebrae from each other by a mechanism other than through the anchor screws, as the anchor screws are moved, under the force of the separation, away from each other, and then retaining the adjacent vertebrae in the spaced apart condition with the anchor screws after the adjacent vertebrae have been separated.

2. A method according to claim 1, wherein the step of separating the adjacent vertebrae from each other includes engaging the intervertebral space between the adjacent vertebrae with a distracter instrument after the anchor screws have been secured to the vertebrae and the frame.

3. A method according to claim 1, including securing two anchor screws into two adjacent vertebrae.

4. A method according to claim 1, including securing three anchor screws, one to each of three adjacent vertebrae.
